(19) United States
(12) Patent Application Publication
(10) Pub. No.: US 2021/0282916 A1
(43) Pub. Date: Sep. 16, 2021

(54) A transapical anatomical stent to repair ascending aorta and hemi arch

(71) Applicant: Cuntao YU, Beijing (CN)

(72) Inventors: Cuntao YU, Beijing (CN); Wei GAO, Beijing (CN)

(21) Appl. No.: 17/257,855

(22) PCT Filed: Jan. 15, 2019
(86) PCT No.: PCT/CN2019/071753

§ 371 Date: Jan. 5, 2021

(30) Foreign Application Priority Data
Sep. 12, 2018 (CN) 201811062289.4

(51) Int. Cl. A61F 2/07; A61F 2/90; A61F 2/95

(52) U.S. Cl. CPC A61F 2/07; A61F 2210/0014; A61F 2/95; A61F 2/90

(57) ABSTRACT

This is an ascending aorta and hemi arch repair stent graft anchored by innominate artery. The graft includes two basic parts: ascending aortic segment and innominate artery segment. The innominate artery portion of the stent graft is firstly released, then the main stent graft is continuously released. The distal end of the main stent graft is against the distal end of the aortic arch, and the main stent graft covers the primary tear location. The proximal stent graft should be positioned precisely above the sinotubular junction. The longitudinal metal beam structure with temperature-dependent shape memory alloy extending along the greater and lesser curvature of the aortic arch would fit to the aortic anatomic morphology perfectly. The graft is secured to the aorta by means of both innominate artery stent graft anchoring and temperature-dependent longitudinal metal beam. The invention is aimed at the patients with type A aorta dissection who cannot undergo the early surgery in the acute phase, providing opportunities for follow-up surgical treatment.

### TECHNOLOGY FIELD

The invention belongs to the field of medical instruments, and specifically relates to the interventional treatment field of aortic stents.

### BACKGROUND

Different from the coronary artery stent, the aortic stent is a large vascular stent with a major purpose to close the aortic tear, open the true lumen, and restore blood flow. So far, such stents are mostly used in the aortic arch and the descending aorta.

Aortic dissection occurs when there is an injury to in the innermost layer of aorta allows blood flow between the layers of the blood vessel wall (dissection). In most cases, there will be severe tearing chest pain or back pain. After the aortic dissection, the true lumen of the aorta is compressed, and blood supply is decreased to organs. This can lead to neurological symptoms, heart failure, mesenteric ischemia, hepatic and kidney dysfunction, paraplegia, etc. The mortality rate of aortic dissection is extremely high.

Aortic Dissection is Classically Divided into Two Types (Stanford Classification):

Stanford classification divides aortic dissection into two types, A and B. It is based on whether the dissection involves the ascending aorta.

Type A aortic dissection (TAAD)—All dissections involving the ascending aorta (surgery is usually recommended).

Type B aortic dissection—All dissections are limited to the descending part of aorta and may extend into the abdomen, but not affect the ascending aorta (nonsurgical treatment is usually recommended).

At present, the mature products in market are mainly aimed at solving the aortic B-type aortic dissection, or a small part of the A-type dissection, that is, the aortic dissection involving the aortic arch and the descending aorta.

Some stent products for ascending aorta have also appeared recently, but all these products are still in clinical trials and have not officially entered clinical use.

The characteristics and main technical limits of current mature products of aortic stents include:

1. The stents are retrogradely inserted through the peripheral arteries (trans femoral artery or radial artery) where the operation site is far from the ascending aorta. Therefore, the management during procedure is complicated and demanding, placing the surgeon in critical condition of implanting the device in the false lumen of the aortic dissection, leading to aorta rupture and even death;

2. The stents need to be anchored to the layered aorta wall, which increases the risk of aorta rupture that is already involved in the dissection, leading to death in some cases. Thus, the indications of surgery are greatly limited;

3. Cervical bypass surgery may be necessary since stent covered the common carotid artery and the left subclavian artery.

Other current technical solutions are to place a descending aorta stent directly in the ascending aorta part with a shorter (because the ascending aorta is short in length) and wider stents (because the diameter of the aorta dilates after dissection and is wider than descending aorta dissection). However, because they are not specifically targeted design, those products could not provide an ideal solution.

The main design problems that have not been solved by existing technologies are:

1. There is no preferable anchoring method. According to existing international guidelines, it is recommended that the aortic stent requires at least 2 cm of sufficient anchoring zone. However, the room for anchoring is generally insufficient after dissection. Meanwhile, the currently available stents on the market are anchored by the existing corolla structure or barb device, which may result in a fatal secondary injury to the ascending aortic dissection. Therefore, it is not suitable for TAAD;

2. Stent implantation may induce coronary perfusion dysfunction. The present way of anchoring stents may damage the endothecium of coronary artery orifice by anchoring them to the aortic sinus of the ascending aorta, thereby hampering coronary perfusion and causing serious consequences such as myocardial ischemia;

3. The diameter of the existing stent is not compatible to the diameter of the ascending aorta after dissection:

4. The long axis of the ascending aorta has a certain curvature and a short length. Existing stent products cannot match the anatomical curvature of the ascending aorta;

5. All the current patents or experimental stents are the ultimate treatment options for TAAD, though with the potential risk of serious complications.

The existing patents are exploring the possibility for the treatment of TAAD, but they are limited and different from this invention in clinical indications, usage and the method of anchoring.

### SUMMARY OF THE INVENTION

The invention is aimed at the patients with acute TAAD namely involving the ascending aorta, which is the most common type.

Open thoracic surgery is commonly recommended to be done within 24-48 hours for patients with acute TAAD considering of its extremely high mortality rate. Nevertheless, 20% patients may die due to their relative contraindications to the surgery at the acute stage. Our invention is focus on the patients who are not suitable for thoracotomy in the acute phase.

The main cause of death in patients with acute TAAD is instantaneous aortic rupture.

The reason why patients with acute TAAD cannot undergo the early surgery in the acute phase is what this invention is going to solve. They are listed as follows:

1. Ascending aortic dissection progresses to the aortic root, involving heart failure.

2. Ascending aortic dissection progresses to the aortic root, involving coronary orifice resulting in acute myocardial ischemia or infarction;

3. Aortic dissection progresses to aortic arch and brachiocephalic artery, seriously affecting the blood supply of the brain and spinal cord, resulting in serious neurological symptoms, such as paraplegia and limb hemiplegia:

4. Severe hepatic and renal dysfunction due to hepatic and renal flow insufficiency after aortic dissection spreads to the distal part of the descending aorta;

5. Sever acidosis due to severe inferior limb perfusion dysfunction after aortic dissection spreads to lower extremities.

For such patients who cannot have the emergency surgery, in order to prolong the survival time while waiting for surgery, we design the stent.

Main clinical issues to be solved by the invention:

1. For those high-risk patients, close the proximal tear of aortic dissection at the first time, which can reduce the amount of blood entering the false lumen, prevent further expansion of the false lumen, avoid the ischemic symptoms and restore the blood supply to the distal organs;

2. High-risk patients who with heart failure, acute myocardial infarction, acute hepatic and renal dysfunction, acute severe neurological complications, acute severe lower limb ischemia and other complications can survive in the acute period under the protection of ascending aortic stent (which prevent dissection rupture) and can have a chance to undergo surgical treatment after the clinical condition is stable;

3. Pericardial effusion often occurs due to thinning and increased permeability of the vessel wall after acute aortic dissection, leading to tamponade even cardiac arrest. The invention stent is placed through the apex approach. During the procedure, pericardial effusion can be drained at the same time, alleviating cardiac compression symptoms and improving cardiac function:

4. Stents are transported directly into the true lumen of ascending aorta dissection via apical delivery (instead the previous conveyors are retrograded through the femoral artery or the radial artery), which prevent the possibility of the conveyance device entering the false lumen fundamentally;

5. There are branch stents placed in the innominate artery on the main stent, then the main stent can be fixed naturally by the shape of its branch into the innominate artery and by the longitudinal metal beam with temperature-dependent shape memory function, without special anchoring devices such as barbs or corollas;

6. The ascending aorta has a unique anatomical curve that the posterior wall is shorter than the anterior wall. However, the existing commercial products cannot meet the anatomy, curvature and deployment precision requirements which unique to the ascending aorta.

To solve the above technical problems and assist those patients with acute TAAD to survive the acute phase with surgical contraindications to the stable stage with the possibility of surgery, the invention provides an ascending aorta and hemi arch repair stent that is inserted through the apical approach and anchored by the shape of branch of the innominate artery.

The invention is realized through the following technical proposal:

This is an ascending aorta and hemi arch repair stent graft anchored by innominate artery. The graft delivered through the apical approach is aimed for acute TAAD with certain surgery contraindications as a bridging strategy on transitional phase. It stands out for the following features: The compressed stent is placed in the delivery system, and the innominate artery portion of the stent graft is firstly released through the apical approach. Thereafter, the main stent graft is continuously released. The distal end of the main stent graft is against the distal end of the aortic arch. As the stent graft released to cover the primary tear location, the closure of the primary intimal tear leads to the expansion of the true lumen, elimination of false lumen and recovery of blood flow. The proximal stent graft should be positioned precisely above the sinotubular junction (STJ). The longitudinal metal beam structure with temperature-dependent shape memory alloy extending along the greater and lesser curvature of the aortic arch would fit to the aortic anatomic morphology perfectly after being released 10-15 minutes at the body temperature of 37° C. The graft includes two basic parts: ascending aortic segment and innominate artery segment. The graft is secured to the aorta by means of both innominate artery stent graft anchoring and temperature-dependent longitudinal metal beam, making it well-fitting against ascending aortic wall. The stent graft is composed of multiple sets of “W”-zigzag mesh stents. The diameter of the ascending aortic stent is 20% greater than that of the dissected ascending aorta, and the length of the ascending aortic stent segment is no more than 10 cm. The coating material of the stent is polytetrafluoroethylene (PTFE) with superior elastic properties.

Preferably, the length of the ascending aortic stent segment is 7-10 cm.

Preferably, the stent is all covered without a bare area.

Preferably, the stent has no crown-shaped opening, and the proximal end of the graft is flat.

Preferably, the multiple sets of double “W”-zigzag mesh stents of the ascending aortic stent segment are three sets, and the multiple sets of “W”-zigzag mesh stents of the innominate artery stent segment are two sets.

Preferably, the distal end of the stent graft has a circumferential radius-support design within PTFE cover.

Advantages and positive effects of the invention:

1. A safer anchoring method. The stent is anchored by adapting the special anatomical branch structure of the innominate artery and by the temperature-dependent shape memory metal beam.

2. A safer and more convenient apical approach. For one thing, through apical approach, antegrade access into the ascending aorta has the advantage of more accurate anchoring of the stent. Meanwhile, because of the apex is closer to the innominate artery with a preferable access angle, the delivery system approach into the innominate artery from the apex will be smoother than any other access approaches. Since the previous techniques were retrogradely through the peripheral arteries (e.g. femoral artery and radial artery), accurate anchoring is still a practical problem due to its long convey distance, which limits the stent development of the ascending aorta all the time. For another, there is slim chance that the stent entering the false lumen of the dissection since the delivery system straightly pass through the left ventricle via aortic valve into the ascending aorta, providing higher safety. Whereas, there is a high likelihood that traditional delivery methods from peripheral arteries enter the false lumen. Moreover, while the stent is being transported, pericardial effusion drainage can also be performed through the apical incision, which can effectively improve the heart function.

3. A specific curvature. In accordance with the unique physical curvature of the ascending aorta, the stent is adjusted by the temperature-dependent memory metal beam along with the greater and lesser curvature and stretches into the innominate artery. The memory metal beam automatically deforms to a personalized shape for the ascending aorta after 10-15 minutes at 37° C. body temperature. Since the length of the lesser curvature of the posterior wall is shorter than that of the greater curvature of the anterior wall in ascending aorta, the deformed memory metal beam covered with the extensible PTFE coating could provide an excellent shape support for the ascending aorta without any damage to the ascending aorta.

4. A wider diameter. According to our current research results, the ascending aorta stent need to extend more than 120% of the diameter of the dissected ascending aorta. However, the stents available now are designed for descending aorta dissection which diameter is only 110% length of descending aorta. It is far from the diameter needs of ascending aorta.

5. A targeted length. In accordance with shorter and physical features of the ascending aorta, the length of the invention stent is 7-10 cm. On the contrary the length of the current stent is rarely below 10 cm.

6. An individualized stent. According to our current research data on the anatomical location of the innominate artery, individualized angle innominate stent will be designed in the future by different temperature-dependent memory metal beam angles.

7. A non-aggressive stent to the coronary perfusion. The invention stent has a flat ending at the proximal end without any crown-shaped structure. The stent proximal ending parallels with the STJ level. Therefore, the coronary artery opening will not be affected in the aortic sinus.

8. A greater coverage. The invention stent is designed as a full-length film covered stent graft, no metal exposed area.

9. A continuable ending. The distal end of the stent continued by a naturally opened PTFE membrane. It can be further connected to the aortic arch stent for following interventional treatment in the long run.

10. An acceptable surgical complication on a certain degree. A small amount of endoleak is resolvable in the subsequent surgical treatment, since the invention stent is a transitional and bridging treatment for the aortic dissection in the acute phase.

### BRIEF DESCRIPTION OF THE DRAWINGS

FIG. 1. The delivery device of the invention enters the ascending aorta through the left ventricle and the apical incision;

FIG. 2. The delivery device of the invention reaches the innominate artery in the true lumen of the ascending aortic dissection;

FIG. 3. After adjusting and determining the direction, the invention continue to release the ascending aorta part of the stent;

FIG. 4. The morphology of the ascending aorta stent of the invention after being completely released;

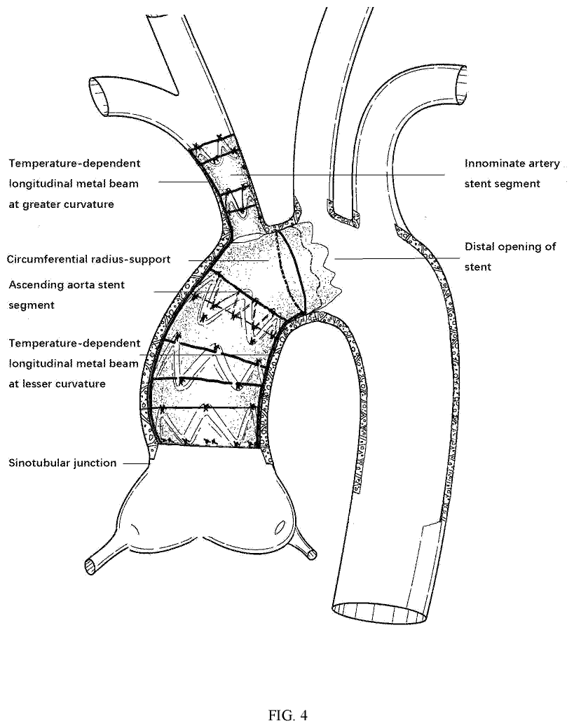

FIG. 5. The alternative 1 of the ascending aorta stent of the invention;

FIG. 6. The alternative 2 of the ascending aorta stent of the invention;

FIG. 7. Schematic diagram of the anatomy of the aorta.

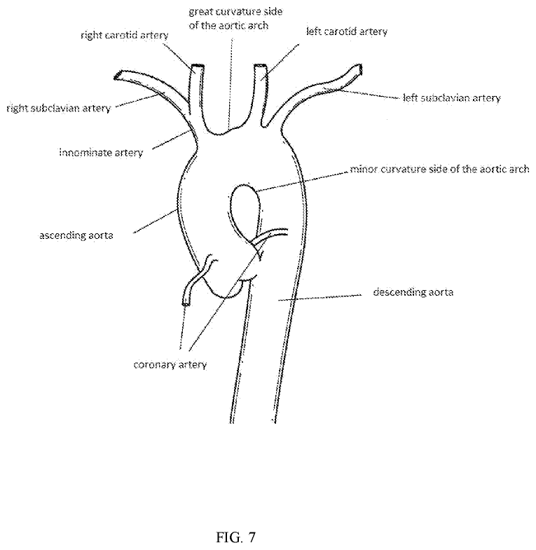

### DETAILED DESCRIPTION OF THE PREFERRED EMBODIMENTS

The technical strategy of the present invention is further described below in the context of a specific case.

An ascending aorta and hemi arch repair stent graft anchored by innominate artery. The graft delivered through the apical approach is aimed for acute TAAD with certain surgery contraindications as a bridging strategy. It stands out for the following features: The compressed stent is placed in the delivery system, and the innominate artery portion of the stent graft is firstly released through the apical approach. Thereafter, the main stent graft is continuously released. The distal end of the main stent graft is against the distal end of the aortic arch. As the stent graft released to cover the primary tear location, the closure of the primary intimal tear leads to the expansion of the true lumen, elimination of false lumen and recovery of blood flow. The proximal stent graft should be positioned precisely above the sinotubular junction. The longitudinal metal beam structure with temperature-dependent shape memory alloy extending along the greater and lesser curvature of the aortic arch would fit to the aortic anatomic morphology perfectly after being released 10-15 minutes at the body temperature of 37° C. The graft includes basically two parts: ascending aortic segment and innominate artery segment. The graft is secured to the aorta by means of both innominate artery stent graft anchoring and temperature-dependent longitudinal metal beam, making it well-fitting against ascending aortic wall. The stent graft is composed of multiple sets of “W”-zigzag mesh stents. The diameter of the ascending aortic stent sizes is 20% greater than that of the dissected ascending aorta, and the length of the ascending aortic stent segment is no more than 10 cm. The coating material of the stent is polytetrafluoroethylene (PTFE) with superior elastic properties.

The length of the ascending aortic stent segment is 7-10 cm.

The stent is all covered without a bare area.

The stent has no crown-shaped opening, and the proximal end of the graft is flat.

The multiple sets of “W”-zigzag mesh stents of the ascending aorta stent segment are three groups, and the multiple sets of “W”-zigzag mesh stents of the innominate artery stent segment are two groups.

The distal end of the stent graft has a circumferential radius-support design within PTFE cover.

The specific operation process is as follows:

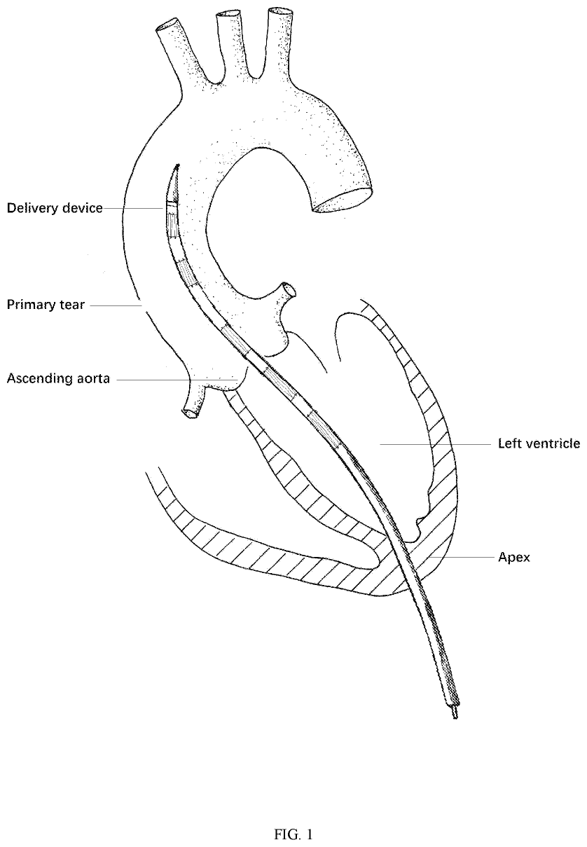

As shown in FIG. 1, the stent graft is placed in a compressed state in the delivery system. An apical incision is made and pericardial effusion can be drained at the same time. A purse-suture is made at left ventricle apex. Puncture the apex, then place the sheath and guide wire. The delivery system is directed by the guide wire.

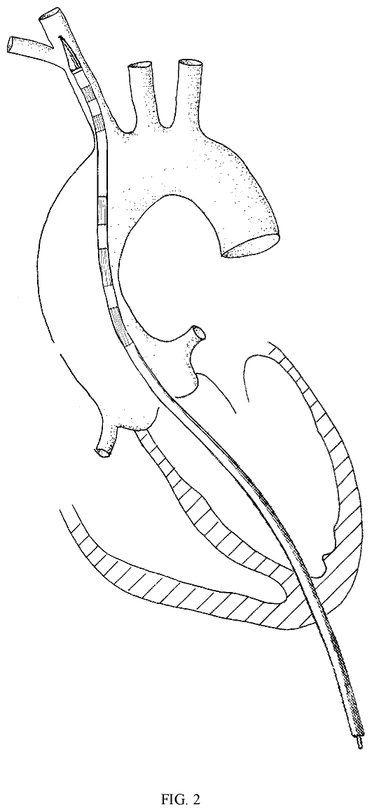

As shown in FIG. 2, the delivery device directs into the innominate artery along the true lumen of the aortic dissection.

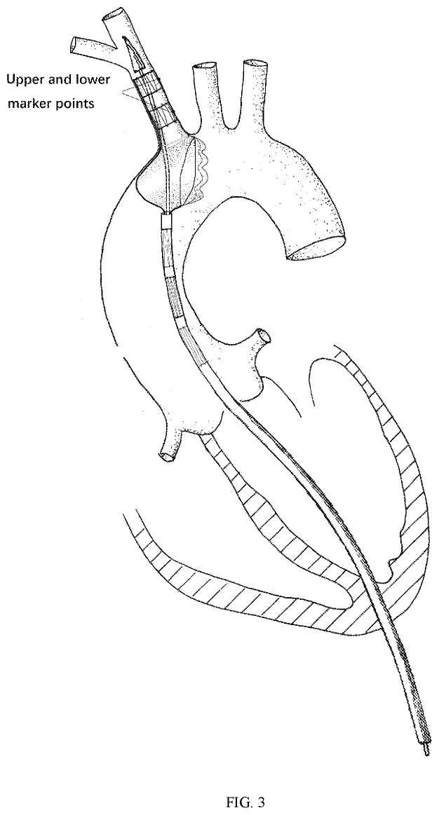

As shown in FIG. 3, the upper and lower sets of marker points on the distal side of the innominate artery stent graft are used to adjust the direction of the main graft (two marker points per set; the proximal side are with memory wires). When the upper and lower sets of markers on the distal end of the innominate artery are doubled into one point, the direction is appropriate and the operation can be continued. Next, continue to release the main stent graft. When the distal opening of the main stent is released, the direction would be reconfirmed. As the stent graft released to cover the primary tear location, the closure of the primary intimal tear leads to the expansion of the true lumen, elimination of false lumen and recovery of blood flow. When completed, the proximal end of the main stent graft terminates is just above the STJ.

The temperature-dependent shape memory alloy longitudinal beam is stored in the delivery system at room temperature. However, it will adjust to the patient's unique aortic anatomy after 10-15 minutes at 37° C. body temperature to ensure perfect anchoring and stability.

Finally, the delivery system is detached from the aortic stent graft and removed from body.

Alternative Design to the Aforementioned:

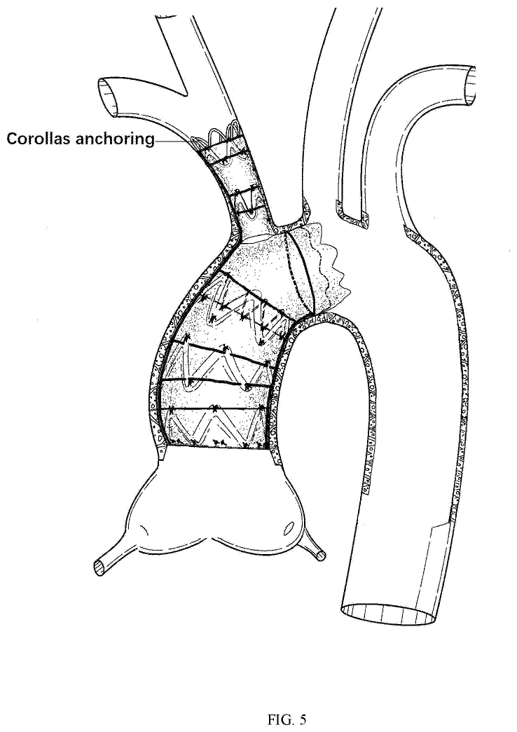

Alternative 1: The distal portion of the innominate artery stent graft is designed a crown-shaped anchoring device performing the anchoring function together with other two methods. This is because the distal part of the innominate artery is rarely involved in acute TAAD and crown structure would not aggravate the condition of aortic dissection. (FIG. 5)

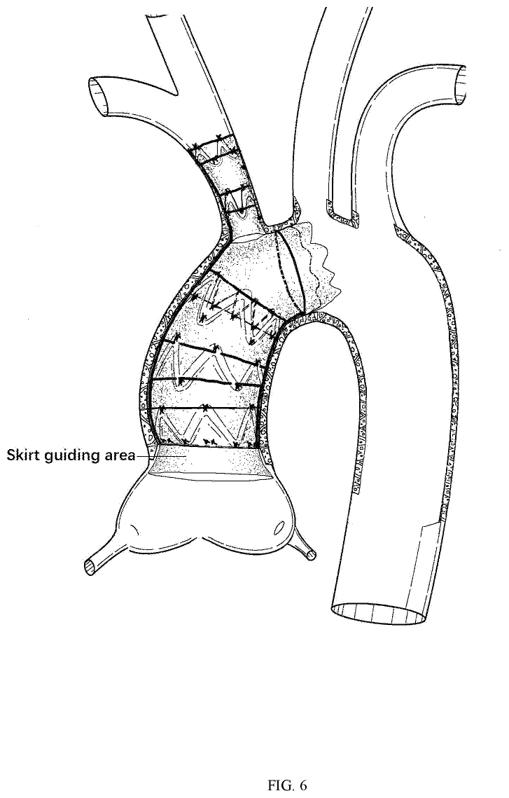

Alternative 2: The proximal end of the main stent graft with a PTFE “skirt” structure designed to direct blood flow from the aortic valve into the true lumen. The special bugle shape of the “skirt” structure could also decrease the probability of type I endoleak to the greatest extent, improving the success rate of the operation. At the same time, the bugle shape can effectively prevent the “skirt” inversion and avoid blocking the ascending aortic opening. In addition, the bugle structure can further eliminate the false lumen and protect the coronary artery orifice to increase coronary perfusion blood flow. (FIG. 6)

Alternative 3: The delivery device passes through the main stent segment rather than innominate artery stent segment, and the innominate artery stent segment is placed separately. When the distal end of the main stent segment is aligned with the aortic arch, the innominate artery corresponding stent segment is released. Subsequent operations are the same as described above. This method of release sequence is more difficult and may require another arterial approach assistance.

As described above, these are only preferred embodiments of the invention, but the protection scope of the invention is not limited to this. Anyone familiar with this technical field can easily think of changes or substitutions within the technical scope disclosed in the present invention, which should be covered within the protection scope of the invention. Therefore, the protection scope of the invention should be subject to the protection scope of the claims.

1. This is an ascending aorta and hemi arch repair stent graft anchored by innominate artery; the graft delivered through the apical approach is aimed for acute TAAD with certain surgery contraindications as a bridging strategy on transitional phase; it stands out for the following features: the compressed stent is placed in the delivery system, and the innominate artery portion of the stent graft is firstly released through the apical approach; thereafter, the main stent graft is continuously released; the distal end of the main stent graft is against the distal end of the aortic arch; as the stent graft released to cover the primary tear location, the closure of the primary intimal tear leads to the expansion of the true lumen, elimination of false lumen and recovery of blood flow: the proximal stent graft should be positioned precisely above the sinotubular junction; the longitudinal metal beam structure with temperature-dependent shape memory alloy extending along the greater and lesser curvature of the aortic arch would fit to the aortic anatomic morphology perfectly after being released 10-15 minutes at the body temperature of 37° C.; the graft includes two basic parts: ascending aortic segment and innominate artery segment; the graft is secured to the aorta by means of both innominate artery stent graft anchoring and temperature-dependent longitudinal metal beam, making it well-fitting against ascending aortic wall; the stent graft is composed of multiple sets of “W”-zigzag mesh stents; the diameter of the ascending aortic stent is 20% greater than that of the dissected ascending aorta, and the length of the ascending aortic stent segment is no more than 10 cm; the coating material of the stent is polytetrafluoroethylene (PTFE) with superior elastic properties.

2. According to the claim 1, the length of the ascending aortic stent segment is 7-10 cm.

3. According to the claim 1, the stent is all covered without a bare area.

4. According to the claim 1, the stent has no crown-shaped opening, and the proximal end of the graft is flat.

5. According to the claim 1, the multiple sets of double “W”-zigzag mesh stents of the ascending aortic stent segment are three sets, and the multiple sets of “W”-zigzag mesh stents of the innominate artery stent segment are two sets.

6. According to the claim 1, the distal end of the stent graft has a circumferential radius-support design within PTFE cover.
